(19) United States
(12) Patent Application Publication
(10) Pub. No.: US 2004/0009160 A1
(43) Pub. Date: Jan. 15, 2004

(54) Bioactive food complex, method for making bioactive food complex product and method for controlling disease

(76) Inventors: Daniel F Villamar, Milwaukee, WI (US); David J W Moriarty, QLD (AU)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/312,039

(22) PCT Filed: Jun. 22, 2001
(86) PCT No.: PCT/US01/16489

(51) Int. Cl. A23K 1/165; A23K 1/17; A61K 45/00; A61K 31/365

(52) U.S. Cl. 424/93.46; 424/442; 514/473

(57) ABSTRACT

A bioactive food complex product, method for preparing a bioactive food complex product and method for controlling disease using probiotics and quorum sensing inhibitors such as inhibitory furanones and other bioactive compounds included in both the continuous and dispersed phases of a bioactive food complex product. The product is comprised of a solids-in-oil or an oil-in-solids emulsion forming a first emulsion that is itself emulsified in polymer forming oil-in-polymer or solids-in-polymer emulsion complex. The bioactive complex is formed of two emulsions with the first emulsion comprising the dispersed phase and a hydrocolloid polymer serving as the continuous phase. The second emulsion complex is then crosslinked to form a physically stable matrix. The bioactive food complex or the first emulsion of the bioactive food complex then serve to deliver different bioactive components including probiotic bacteria and quorum sensing inhibitor molecules to the digestive tract and environment of animals such as shrimp or fish or other livestock raised commercially to effectively control bacterial disease by a novel combination of mechanism including: competitive exclusion, direct inhibit, digestion of cell-to-cell signaling molecules and direct inhibition of homoserine lactone and (acyl) homoserine lactone regulated processes of pathogenic bacteria. Thus, effective disease prevention and control is accomplished through the novel combined delivery and use of probiotic bacteria and quorum sensing inhibitory furanones.

### FIELD OF THE INVENTION

The present invention relates to a bioactive food complex product, method for preparing a bioactive food complex product and method for controlling disease using probiotics and quorum sensing inhibitors such as inhibitory furanones and other bioactive compounds included in both the continuous and dispersed phases of a bioactive food complex product.

### DESCRIPTION OF THE BACKGROUND

Aquaculture of shellfish and finfish provides high-value food products for human consumption and has been the most rapidly growing sector in international agribusiness. Continued progress in aquaculture is limited by: (1) the lack of adequate commercial feeds during critical hatchery and nursery phases, and (2) devastating losses to disease in all production phases particularly in shrimp farming.

Hatchery and nursery operations typically depend on supplies of fresh and live food organisms such as squid, polychaete worms, Artemia biomass, Artemia nauplii and microalgae to produce aquaculture seedstock for grow-out and production aquafarms. These foods typically carry high bacterial loads, which can include pathogens such as Vibrio bacteria, and could be vectors for viral disease transmission. Use of fresh and live food organisms increases the risk of disease in the hatchery and these disease agents can be transported to nursery and grow-out facilities via the seedstock.

Massive losses to disease in shrimp farming mostly due to bacteria, especially Vibrio species, and viruses such as White Spot Virus have caused heavy damage to this industry in countries such as China, Thailand, Indonesia, India, Philippines, Panama, Ecuador and others. The high density of animals in hatchery tanks and ponds is conducive to the spread of pathogens. The aquatic environment, with regular applications of protein-rich feed, is ideal for culturing bacteria, many of which can be pathogenic. These problems have been exacerbated because the interactions of microbes, animals and their environment at intensive production scales have been considered primarily from a clinical pathology perspective. When pathogenic bacteria or viruses are detected, farmers apply antimicrobial compounds to the feed and water; many farmers also use antibiotics as prophylactics in large quantities, even when pathogens are not evident. This has lead to an increase in vibrios, and presumably other bacteria, having multiple antibiotic resistance and being more virulent pathogens. Pathogens include species of Vibrio harveyi, V. parahaemolyticus, V. splendidus, V. mimicus, V cholerae, V alginolyticus, V. anguillarum, and others.

The use of beneficial bacteria (probiotics) to displace pathogenic bacteria by competitive processes and by direct inhibition is a better remedy than administering antibiotics. Probiotics can be used to control diseases caused by Vibrio and other Gram negative bacteria and to control other diseases of fish and shrimp including those caused by Aeromonas species and Gram positive pathogens, including Streptococcus, Camobacterium etc.

These are now used in the aquaculture industry, but their efficacy is variable; many products are not designed for aquaculture or-are not specific for the target animals or the pathogens they are supposed to control. In other words, they do not meet the definition of a probiotic: a culture of naturally occurring live microorganisms that when eaten confers a health benefit on the animal.

Generally, the probiotics used are applied to the water or in some cases they were mixed with feed immediately before use. As high temperatures are used in dry, pelleted feed manufacture, living bacteria are killed, so the probiotics could not be incorporated during manufacture of many pelleted and most extruded feeds.

WO 9629392 describes a method for inhibiting a homoserine lactone regulated process in certain Gram negative bacteria, especially Vibrio species, by using a furanone; however, no mechanism was proposed for delivery of the furanone that was practical in commercial scale aquaculture.

In the hatchery, little has been done to efficiently control the microbial populations through the food and water. Bioencapsulation i.e., the enrichment of live food organisms by feeding them on suspensions, emulsions or other preparations containing desired payload compounds for subsequent feeding to larval fish and shrimp, has been used to increase and improve the nutrient content of live food organisms such as the brine shrimp, Artemia sp. (Lavens et al., Aquaculture and Fisheries Management (1992)). This technology has also been used to deliver bioactive compounds such as antibiotics (Dixon et al. 1995, Journal of Aquatic Animal Health 7:42-45) and bacteria (Gomez-Gil et al. 1998, Applied and Environmental Microbiology 64(6): 2318-2322) to aquaculture species. Bioencapsulation is an interesting process that provides a vehicle for delivery of nutrients and bioactive compounds within food organisms to target aquaculture species. However, bioencapsulation adds complexity to the already labor-intensive process of culturing both the live food organisms and the target species raised in commercial hatcheries.

Commercially available dry larval feed products including protein-crosslinked microcapsules, spray-dried agglomerates, freeze-dried particles and flakes have been marketed as live-food replacements or dietary supplements for larval fish and shrimp. However, survival and/or growth of larval fish and shrimp fed on these dry larval feeds in the absence of live food organisms are typically poor. Consequently, marine fish and shrimp hatcheries can reduce but not eliminate the use of live food organisms for seedstock production.

Problems associated with the use of dry larval feed products for shrimp and fish include: (a) physical instability in water i.e., feed particles decompose rapidly after adding to culture tank; and (b) high rate of nutrient leaching i.e., water-soluble organic matter escapes from intact feed particles/microcapsule into tank water. Physical decomposition and leaching of commercial larval feeds contribute to poor water quality. Poor water quality is a source of stress on larval fish and shrimp and can reduce health and survival rate in the hatchery by promoting growth of pathogens.

Decomposition and leaching of larval feed types is reduced by: (a) harsh drying methods that harden feed particles making them relatively water-insoluble; (b) enrobing or encapsulating feed nutrients in a water-insoluble coating or matrix; or (c) by using feed ingredients with low water solubility in larval feed preparations. However, these methods improve water stability and reduce leaching at the expense of bioavailability of nutrients to larval fish and shrimp that have poorly developed digestive systems and can not obtain adequate nutrition from these feed types.

Protein cross-linked microcapsules (British Patents 79437454 and 2103568) are sold commercially as a dry powder to replace live-food organisms in the diet of larval shrimp. However, these capsules lose large amounts of soluble nutrients by leaching and cannot completely replace live algae and Artemia in shrimp larviculture.

WO 87/01587 describes the formation of lipogel particles whereby a nutritional or pharmacologically active payload is entrapped in liposomes and the liposomes are encapsulated in a hydrocolloid matrix. The liposomes would effectively contain small, water-soluble compounds within a phospholipid microsphere and the hydrocolloid matrix would protect the liposomes. However, it is unlikely that lipogel particles could contain sufficient nutrient density to comprise a complete larval feed due to the small volume of the liposome payload and to the relatively fragile phospholipid membranes which would likely rupture when mixed with other nutrient feed ingredients required for a complete larval feed.

Villamar and Langdon (Marine Biology, 115(4): 635-642 (1993)) describe complex microcapsules (CXMs) that greatly reduced leaching of low molecular weight, water-soluble compounds from a larval feed. CXMs were made by embedding lipid-wall microcapsules (LWMs) containing the aqueous payload material into larger gel beads containing nutrient feed ingredients and composed of sodium alginate and gelatin ionically crosslinked with calcium. LWMs could retain small water-soluble molecules within CXMs, but their payload volume was small and manufacturing process difficult.

Larval feed CXMs serve to solve some of the problems of physical water stability and nutrient loss by leaching, but do not address delivery of bioactive compounds or control of disease. Bioencapsulation serves to improve the nutrient value of live food organism and permits delivery of bioactive compounds to target species, but the art adds more complexity and expense to the already complex process of raising larval fish and crustaceans.

U.S. Pat. No. 5,698,246 builds on the CXM concept developed by Villamar and Langdon (Marine Biology, 115(4): 635-642 (1993)) by simplifying the process to eliminate the complexity of forming LWMs by coating or enrobing the dry nutrient feedstuff with lipid such as fishoil then encapsulating the coated or enrobed feedstuffs along with Bacillus sp. bacteria as “endo-probiotics”. The payload mixture of enrobed feedstuff and bacteria are encapsulated in a hydrocolloid matrix such as sodium alginate ionically crosslinked with calcium. The encapsulated complex is then stabilized as a suspension in a preservative liquid medium containing probiotic bacteria as “ecto-probiotic”. In this liquid larval feed endoprobiotics are designed to enter the GI tract of target larval shrimp or fish along with the nutrient payload and the ectoprobiotics are designed to remain in and colonize the water in which the shrimp or fish are raised.

While U.S. Pat. No. 5,698,246 represents a significant improvement over conventional preparations of feeds for aquaculture by the invention of a novel liquid feed type, it does not provide a method for controlling disease nor does it advance the art of making CXMs, rather, it substitutes LWMs with a simple coating or enrobing process of payload materials and provides a method for delivering probiotic bacteria without describing a method for control of diseases in aquaculture.

U.S. Pat. No. 5,776,490 is a modification of the CXM concept with LWMs contained within complex, cross-linked protein microcapsules rather than within a hydrocolloid matrix of ionically crosslinked calcium alginate and gelatin. The invention has application in the delivery of nutrients and drugs to larval finfish in aquaculture but does not provide a method for disease control.

Phase-1 USDA/SBIR Grant No. 93-33610-8500 (1993) describes an approach that serves to form a water-stable aquatic feed that is processed under low temperature, below 85° C., to optimize nutrient bioavailability to aquatic animals. The water-stable feed is made by mixing a slurry containing feed ingredients and sodium alginate in water then either cold-extruding or pouring the mixture into molds to form gel strips that are crosslinked with calcium chloride and dried at low temperature. Alternatively, the art describes vacuum drum drying the slurry to form thin sheets that are crosslinked with calcium chloride. The art serves to use low-temperature processing to avoid heat damage to nutrients prepared for aquatic animals, but does not address the delivery of bioactive compounds or control of disease.

WO 95/28830 describes an “ambient-temperature process” i.e., 3°-156° C., that comprises mixing alginate, feed ingredients and water to make a slurry that is exposed to divalent cations to form a water-stable aquatic feed pellet. WO 95/28830 does not appear to contain any improvements over compositions and methods.

U.S. Pat. No. 6,024,983 describes a microcapsule composition for delivery of bioactive compounds in medical applications of potentiating the immune response animals. The invention consists of a mixture of a primary biocompatible microcapsules containing a bioactive agents encapsulated in secondary biocompatible microcapsules containing a bioactive agents. The invention provides for immune response to antigens of animal or human diseases ranging from influenzae antigen to gonorrhoea antigen. The applications of the invention target use of such bioactive compounds in diseases affecting animals with developed immune systems and homoeothermic. This invention however does not have application in shrimp, animals with a non-specific immune system, and does not provide a method for disease control in aquaculture as it is related to the biomedical field.

In another industry, microencapsulation using hydrocolloid microcapsules is used to immobilize biologically active materials including hybridoma cells (U.S. Pat. No. 5,116,747, U.S. Pat. No. 4,942,129) and islets of Langerhans (U.S. Pat. No. 4,806,355). The cells are immobilized in gel beads for biomedical purposes including mass culture in bioreactors to produce commercial quantities of biologics, or for disease treatment by medical implant in human or animal bodies not aquaculture.

In yet another industry application, calcium alginate microencapsulation is used to immobilize biologically active Lactococcus lactis subsp. cremoris bacteria in gel beads for the purpose of cell biomass production for dairy fermentation (Morin et al., Applied and Environmental Microbiology, 58(2):545-550, (1992)).

In a research application, Chevalier and de la Noue (Enzyme Microb. Technol., 9;53-56, (1987)) enhanced alpha amylase enzyme production by immobilizing bioactive Bacillus subtilis bacteria in ionically-crosslinked carrageenan gel beads in an airlift fermenter.

In the animal agriculture industry, Lactobacillus sp., Bacillus sp. and other bacteria are probiotics commonly added to feeds as direct-fed microbial to enhance nutrient digestion by enzymes and to suppress growth of potential pathogenic microorganisms in the gut of livestock, particularly during times of stress. Improvements of livestock health are generally attributed to secretion of antimicrobial substances that inhibit growth of pathogens in the gut and to stimulation of specific and non-specific immune response by the probiotics.

In aquaculture, the delivery of bioactive materials such as probiotics to aquatic organisms, especially to larval forms, can not be readily accomplished by conventional feeds, which decompose rapidly in water, have high-rates of leaching, or have been harshly treated during the manufacturing process. While U.S. Pat. No. 5,698,246 and U.S. Pat. No. 5,776,490 represents improvements in the art of aquatic feeds, especially for larval fish and shrimp, they are variants procedures developed by Villamar and Langdon (Marine Biology, 115(4): 635-642 (1993)) that do not describing a method for control of diseases in aquaculture.

Research with marine fish indicates that several strains of bacteria naturally present in the gut produce inhibitory substances against bacterial pathogens such as Vibrio anguillarum and these beneficial bacteria can adhere to and grow in intestinal mucus of the fish (Olsson et al., Applied and Environmental Microbiology, 58 (2):551-556, (1992)).

The invention described herein provides a composition of matter that is an improvement over Villamar and Langdon (Marine Biology, 115(4): 635-642 (1993)) and provides a method for control of bacterial disease not described elsewhere.

### SUMMARY OF THE INVENTION

The present invention provides the composition of a bioactive food complex for aquatic animals, a process for making a bioactive food complex and a method for feeding the bioactive food complex to aquatic animals including but not limited to crustacean, molluscan, and finfish larval, postlarval, juvenile and adult forms, and a method for controlling bacterial diseases in aquaculture.

The present invention provides a method for incorporating selected probiotics as spores into the feed during manufacture and providing the mechanism to activate and germinate them when they are fed to the animals or added to water in ponds or tanks. Furthermore, this method is combined with a method for controlling pathogens by inhibiting expression of genes that regulate virulence gene activity. This combination of methods is much more effective than each on its own and is commercially feasible. The probiotic bacteria are selected for production of enzymes that degrade both Homoserine Lactones and Acyl Homoserine Lactones produced by Vibrio species and that produce antibiotics to inhibit growth of or kill Vibrio species.

The present invention describes a practical mechanism for delivery of inhibitory furanones to animals raised commercially to inhibit a homoserine lactone regulated processes in Gram-negative bacteria, especially pathogenic Vibrio species. The present invention combines the delivery of those inhibitory compounds with the delivery of probiotic bacteria and other bioactive compounds in animal feed and in animal containment systems.

Thus, the present invention provides a method of controlling and/or preventing diseases in aquatic animals, comprising feeding the aquatic animals a composition comprising at least one probiotic bacteria and at least one inhibitory or regulatory compound. In this method, bacterial pathogenicity may be inhibited by a combination of the following three mechanisms working together

a. Use of probiotic bacteria such as Bacillus species to control pathogens by competitive exclusion such as competition for food and space, and by direct inhibition such as by in situ production of bactericidal or bacteriostatic compounds such as antibiotics against Gram negative and Gram positive pathogens reducing their numbers preventing quorum sensing and the expression of virulence genes;

b. Use of probiotic bacteria such as Bacillus species to inhibit virulence gene expression in Gram negative and Gram positive pathogenic bacteria by the effect of enzymes secreted by the probionts that degrade quorum sensing molecules secreted the pathogens.

c. Delivery of inhibitory or regulatory compounds such as furanones that inhibit regulation of virulence gene expression with Homoserine Lactones and Acyl Homoserine Lactones in Gram negative pathogenic bacteria such as Vibrio species.

In one embodiment of the bioactive food complex, a primary emulsion of solids-in-oil comprised of lipid soluble bioactive compounds such as inhibitory furanones dissolved in lipid forms the continuous phase with dry feed ingredients and other bioactive compounds such as selected probiotic bacteria forming the dispersed phase of the stable emulsion. The stable emulsion, Emulsion-1, provides bioactive components in both the dispersed and continuous phases and indispensable nutrients required for normal survival, growth and development of aquatic animals. Emulsion-1 is itself emulsified as the dispersed phase in hydrocolloid polymer which is then ionically gelled forming the bioactive food complex. In the following disclosure, hydrocolloid is a preferred polymer type but other forms of the continuous phase of the second emulsion can include cross-linked protein or other biodegradable polymer forms. The bioactive food complex contains bacterial spores, vegetative bacterial cells, bacterial cell walls, yeasts cells, yeast extract, yeast cell walls, algal cells, medicaments, enzymes, invertebrate embryos or bioencapsulate invertebrate organisms that provide essential nutrients and/or bioactive compounds that can help improve nutrients absorption and assimilation efficiency, enhance immune response and suppress pathogenic microorganism growth in the digestive tract of aquatic animals and in the water of ponds or tanks or other containment systems in which aquatic organisms are raised.

The bioactive food complex is preserved without drying as a semi-solid, moist paste, not a liquid, composed of microcapsules or beads, or as moist noodles, pellets, sheets or other forms, or can be stored frozen by employing cryopreservatives. The bioactive food complex can be added directly to aquaculture animal containment systems to be eaten by aquatic animals. In one embodiment of the invention, the bioactive food complex or the primary emulsion of the bioactive food complex can be added to pelleted or extruded aquatic feeds as a top-dress coating or enrobing of the pelleted or extruded aquatic feed.

A novel two-emulsion process makes the bioactive food complex. In one embodiment, Emulsion-1 is a solids-in-oil emulsion consisting of bioactive materials and powder nutrient feedstuffs forming the dispersed phase and edible oil containing dissolved lipid soluble bioactive compounds forming the continuous phase. Emulsion-2 is an oil-in-polymer emulsion in which Emulsion-1 serves as the dispersed phase and hydrocolloid polymer serves as the continuous phase. Emulsion-1 contains nutrient feed ingredients and bioactive materials dispersed in oil and it is emulsified in hydrocolloid polymer forming Emulsion-2. In one embodiment of the invention, bioactive materials including inhibitory furanones that are lipid soluble are blended into the continuous phase of Emulsion-1. In another aspect of the invention, bioactive materials such as invertebrate organisms or embryos or vegetative cells of probiotic bacteria that could be ruptured or damaged during formation of Emulsion-1 can be directly embedded into Emulsion-2 by gentle mixing. Emulsion-2 is then ionically crosslinked or complexed to form a physically stable matrix that binds the bioactive materials and nutrients of Emulsion-1 and the intact bioactive materials embedded in Emulsion-2. During the ionic gelation process, the bioactive food complex can take the shape of microcapsules or beads, noodles, pellets, sheets or other geometric forms.

The edible oil used as the continuous phase of Emulsion-1 is either a fish oil, a refined fish oil product or a combination of fish oil, fish oil product and vegetable oil. Typical oils include menhaden fish oil, salmon oil, anchovy oil, sardine oil, tuna oil, mackerel oil, capelin oil, squid oil, pollack oil, cod liver oil, dietary fish oil supplements such as Promega (Warner-Lambert Co.), soybean oil, safflower oil, corn oil, palm-kernel oil and other edible oils. In addition, the oil or oil mixture has added inhibitory furanones, lecithin, cholesterol, emulsifying agent such as Santone (Van Den Bergh Food Ingr.), antioxidant such as ethoxyquin, fat-soluble vitamins A, D, E and K, beta-carotene, and astaxanthin pigment.

After the inhibitory furanones and other lipid soluble bioactive compounds, lecithin, cholesterol, emulsifying agent, antioxidant, fat-soluble vitamins and astaxanthin are blended into the oil, the dry powder feed ingredients are emulsified into the oil mixture forming Emulsion-1, which is a stable emulsion consisting of solids-in-oil. In a preferred aspect of invention, the ratio of solids to oil ranges from about 0.01:1 to 100:1, preferably 0.1:1 to 3:1. Thus the composition of Emulsion-1 can range from one of solids-in-oil to oil-in-solids depending on the relative concentrations of solids and oil.

Both types of emulsions are within the scope of the invention. In the following disclosure, when the emulsion is described as a solids-in-oil emulsion it is to be understood that the description also applies to the oil-in-solids emulsion and that bioactive compounds are contained in both the dispersed and continuous phases of both types of emulsions, unless indicated otherwise.

In a preferred embodiment of the invention, bioactive materials including bacterial cell walls, bacterial cells or spores, yeasts, yeast cell walls, yeast extract, algal cells, medicaments or enzymes are included in the solids phase of Emulsion-1. The oil phase of Emulsion-1 serves as medium to carry lipid soluble bioactive compounds such as inhibitory furanones, lipid soluble hormones, lipid soluble chemo-attractants or chemo-stimulants or other lipid soluble bioactive compounds and serves as a hydrophobic inner coating for the powder feed ingredients and bioactive materials. Emulsion-1 also serves as a nutrient source for aquatic animals by providing essential fatty acids, lipid-soluble nutrients and vitamins and pigments.

The present invention, unlike liposomes, LWMs or known coated/enrobed matter, Emulsion-1 is a novel solids-in-oil or oil-in-solids emulsion that contains important bioactivity in both the continuous and dispersed phases, is simple to manufacture, stable, can provide a barrier to reduce the leaching of water-soluble molecules from the powdered nutrients and from the bioactive materials into the aquatic environment and is not limited by the small payload volume of liposomes or LWMs. Emulsion-1 is physically and chemically different than the simple coating or enrobing process of U.S. Pat. No. 5,698,246 and represents an improvement over the complex microcapsules (CXMs) described by Villamar and Langdon (Marine Biology, 115(4): 635-642 (1993)).

Emulsion-1 containing both lipid soluble and solid bioactive materials and feed ingredients is itself emulsified into a hydrocolloid polymer dispersion forming Emulsion-2, an oil-in-polymer or solids-in-polymer emulsion. That is, the dispersed phase of Emulsion-2 consists of Emulsion-1 and the continuous phase of Emulsion-2 consists of hydrocolloid polymer. Invertebrate embryos or invertebrate organisms, vegetative bacterial cells or other materials that can be ruptured or damaged by emulsification can be imbedded in Emulsion-2 by gentle blending. Emulsion-2 is then exposed to ions that serve to ionically crosslink the hydrocolloid polymer. Ionic gelation serves to form a stable matrix of the continuous phase of Emulsion-2 that entraps the dispersed phase of the Emulsion-2 and embedded material forming the bioactive food complex.

In one embodiment, the bioactive food complex forms a semi-solid moist paste and is preserved by decreasing the available water (A_{w}) to below 0.8 in the presence of an antibacterial medium such as calcium chloride or sodium chloride brine and with a mold inhibitor such as glycerol, propylene glycol, proprionic acid or other preservative. Additionally, the bioactive food complex can be stored frozen by employing a cryopreservative such as dimethylsulfoxide (DMSO), glycerol, butylated hydroxytoluene (BHT), sucrose, raffinose, manitol, ethylene glycol, propylene glycol, methanol, 1,2-propanediol, 1,3-butanediol, 2,3-butendiol.

### DETAILED DESCRIPTION OF THE INVENTION

Inhibitory furanones, spores and dried vegetative cells of probionts such as Bacillus species selected for antimicrobial chemical and enzymatic production specific for inhibition of Gram negative pathogens such as Vibrio are incorporated into bioactive food complex during manufacture. The bioactive food complex is then added directly to the foods or animal containment systems of aquatic species in aquaculture. The bioactive food complex and Emulsion-1 of the bioactive food complex are added separately or together as active ingredients of top-dress coatings of conventional animal feeds including pelleted or extruded aquatic feeds.

The Bacillus species are selected on the basis of producing antibiotics to inhibit or kill pathogenic bacteria such as Vibrio. The same or separate Bacillus species are selected on the basis that they produce enzymes that inhibit cell to cell signaling in the pathogen such as Vibrio species by degrading or digesting signal molecules such as Homoserine Lactones (HSL) and (Acyl) Homoserine Lactones (AHL).

In one embodiment of this invention, the inhibitory effect of degrading or digesting signal molecules of pathogens is combined with competition for nutrients and attachment sites in the gut of shrimp, fish, molluscs and other aquatic animals and on their external surfaces such as on the exoskeleton of crustaceans, soft-tissue of molluscs and in the slime layer of fish thus combining lowering of numbers of pathogens such as Vibrio on these surfaces with inhibiting expression of virulence genes.

The combination of heat and pressure during the manufacture of the bioactive food complex and of ordinary animal feed is sufficient to prime (“activate”) the Bacillus spores for germination, providing manufacturing temperature is maintained preferably below 100° C. Germination is further promoted by incorporating soluble contents of microorganisms such as yeast extract into the pellet to provide alanine and other amino acids that stimulate germination upon hydration of the bioactive food complex or feed.

In a preferred embodiment of this invention, the spores and dried vegetative cells of Bacillus species and other bioactive compounds, especially inhibitors of quorum sensing processes regulated by signal molecules such as HSL and AHL in Emulsion-1 or in the bioactive food complex are added to aquatic or extruded feeds as top-dress coatings. Competition and inhibition by Bacillus species probiotics reduces the numbers of Gram negative and Gram positive pathogens in the gut, body surface and/or environment of animals preventing quorum sensing in the pathogens and thus preventing the activation of genes for virulence of these pathogens due to their low numbers. This invention prevents population densities of pathogens from increasing above a minimum threshold for activation of the virulence genes. By keeping pathogen numbers low with probiotics of the invention that compete for nutrients, space and directly kill pathogens, the quorum sensing regulatory genes are inhibited, virulence genes are not expressed and disease does not occur.

The method of the present invention is different from others because the invention provides for a combination of processes or mechanisms that together are most effective. For example, if inhibitory furanones are used alone or the HSL and AHL regulatory molecules are destroyed or digested without competitive exclusion or direct inhibition of pathogens by probionts of this invention, then the growth of pathogens is not prevented, only the expression of virulence genes is prevented, so if the supply of inhibitory furanones is stopped suddenly, for example if the farmer runs out of furanone treated feed, then quorum sensing will occur and virulence factors will be secreted, permitting disease and killing the animals. The Bacillus will have two effects: (1) the enzymes produced by the Bacillus will degrade HSL and AHL produced by the pathogens such as Vibrio and prevent virulence genes from being expressed; (2) the numbers of pathogens such as Vibrio will be low due to inhibition and competition for food and other resources, and thus the low population density means that a quorum is not present, i.e. the pathogen numbers are too low for the concentration of HSL or AHL compounds to be effective. The result will be no expression of virulence genes. No disease.

The probiotics compete for nutrients, and thus inhibit rapid growth of pathogens. On their own, the probiotic bacteria might not be effective when pathogens have genes for resistance to the antibiotics produced by the probionts. In a preferred embodiment of this invention over other procedures, when probiotic and quorum sensing mechanisms are used together a lower number of probiotic bacteria will be effective, making product manufacture simpler and less expensive. Furthermore, it will be far less likely that a pathogen could develop resistance to all the antibiotics produced by the range of Bacillus species used, and also circumvent the effect of enzymes that degrade their signaling molecules.

In the preferred embodiment, Emulsion-1 of the invention is a solids-in-oil emulsion consisting of bioactive materials and powder nutrient feedstuffs forming the dispersed phase and an edible oil preparation containing dissolved lipid soluble bioactive compounds forming the continuous phase. In a preferred embodiment of the invention the oil phase contains inhibitory furanones and other bioactive lipid soluble compounds. The oil preparation is made by mixing into edible oil one or more of the following at about 0.0001-50 weight percent lipid soluble bioactive compounds such as inhibitory furanones, 0.5-5 weight percent emulsifying agent such as Santone (Van Den Bergh Food Ingr.), , about 1-10 weight percent lecithin, about 1-10 weight percent cholesterol, about 0.01-0.05 weight percent antioxidant such as ethoxyquin, about 2,000 to 8,000 IU vitamin-A per kg oil, about 1,000 to 4,000 IU vitamin-D₃ per kg oil, about 2,000 to 8,000 IU vitamin-E (alpha-tocopherol) per kg oil, about 20 to 80 mg vitamin-K (menadione) per kg oil, about 2,000 to 8,000 IU beta-carotene per kg oil, and about 500-2,000 mg astaxanthin per kg oil.

Typical edible oils include refined menhaden fish oil, salmon oil, anchovy oil, sardine oil, tuna oil, mackerel oil, capeline oil, squid oil, pollack oil, cod liver oil, dietary fish oil supplements such as Promega (Warner-Lambert Co.), soybean oil, safflower oil, corn oil, palm-kernel oil and other edible oils. The oil preparation has a concentration of total omega-3 fatty acids greater than about 1 weight percent, eicosapentaenoic acid (EPA) greater than about 0.1 weight percent, and docosahexaenoic acid (DHA) greater than about 0.1 weight percent. To form Emulsion-1 the oil mixture is warmed to about 25° C.-40° C. before adding the powder nutrient feedstuffs and the bioactive materials.

The composition of the solids phase of Emulsion-1, which consists of powder nutrient feedstuffs and bioactive materials, can be modified, as needed to provide indispensable dietary components, energy and bioactive materials required by different aquatic species, different life-cycle stages of a particular species, or by different aquaculture applications such as for routine feeding or for increased fortification and immune system stimulation during times of stress e.g., the transport of postlarvae or fly from hatchery to grow-out ponds. The following composition of the solids phase of Emulsion-1 can be varied to account for the broad range of possible application of the bioactive food complex for animals.

The solids phase of Emulsion-1 is composed of one more more of the following bacterial spores, bacteria cell walls, bacterial cells, including but not limited to Lactobacillus, Bacillus, Streptococcus, Bifidobacterium, Leuconostor, and Alteromonad, at about 0.1-95 weight percent; yeast extract, yeast cell walls, dietary yeast, brewer's yeast, yeasts cells including but not limited to torrula yeast and Phaffia yeast at about 0.1-50 weight percent; algal cell preparations, algal cells including but not limited to Haematococcus, Schizochytrium, Dunelliela, Chaetoceros, Tetraselmis, Skeletonema, Nannochloropsis, Thalassiosira, Phaeodactylum, Isochrysis, Pavlova, at about 1-80 weight percent; medicaments including but not limited to antibiotics such as Sarafin, Romet, Terramycin at about 0.01-50 weight percent; powder feedstuffs, with concentrations adjusted to meet animal's dietary requirements include animal protein products, at about 0-95 weight percent; plant protein products, at about 0-95 weight percent; poultry egg products, at about 0-25 weight percent; cyanocobalamin at about 40-60 mg/kg; D-biotin at about 5-20 mg/kg; D-pantothenic acid at about 250-350 mg/kg; folic acid at about 10-30 mg/kg; L-ascorbyl-2-polyphosphate (STAY-C, stable form of vitamin C) at about 1,000-4,000 mg/kg; myo-inositol at about 3,000-4,000 mg/kg; niacin at about 600-800 mg/kg; p-amino-benzoic acid at about 350-450 mg/kg; pyridoxine hydrochloride at about 40-60 mg/kg; riboflavin at about 125-175 mg/kg; thiamine hydrochloride at about 50-80 mg/kg; choline chloride at about 6,500-7,500 mg/kg.

Emulsion-1 is made by mixing the solids components into the oil mixture in a ratio of about 0.01:1 to 100:1, preferably 0.1:1 to 3:1, and will either be a solids-in-oil or an oil-in-solids emulsion depending on the relative concentrations of the solids and oil components. Emulsion-1 is stable and does not phase-separate for at least 3 hours when kept at 20-25° C.

Emulsion-2 is formed by mixing Emulsion-1 into hydrocolloid polymer at about 5 to 65 weight percent depending on the application of the bioactive food complex. The preferred composition for larval shrimp is about 35-45 weight percent Emulsion-1 in hydrocolloid polymer. The continuous phase of Emulsion-2 is composed of about 0.5-4.0 weight percent sodium alginate or kappa carrageenan polymer formed in deionized water at about 45° C.-85° C. The alginate or carrageenan polymers can be blended with dissolved or dispersed gelatin, zein, polylysine, polyarginine, chitosan, gum accacia, or locust bean gum preparations at about 0.1-3.5 weight percent in water to add sites for proteolytic digestion by aquatic animals and improve gel matrix conformation and binding strength. The ratio of alginate or carrageenan to polypeptides or proteins such as gelatin or to other hydrocolloids or gums such as locust bean gum or chitosan is about 2:1 to about 10:1.

In the case where sodium alginate serves as the continuous phase of Emulsion-2, the aqueous medium is adjusted to about pH 12 to assure that alginate molecules are negatively charged to react with calcium forming the calcium alginate matrix. In the case of kappa carrageenan polymers, potassium is the preferred ion that serves to react with the polymer to form a potassium carrageenan complex. The polymer or polymer blend comprising the continuous phase of Emulsion-2 serves to encapsulate or entrap Emulsion-1 when complexed with calcium or potassium.

In another aspect of the invention, relatively fragile bioactive components such as invertebrate embryos or invertebrate organisms are embedded in Emulsion-2 before forming an ionic gel matrix by gently blending the fragile materials into Emulsion-2 at about 1-50 weight percent. The fragile materials can include any suitable live, frozen or lyophilized metazoan, protozoan or other microorganisms or plant or animal tissue providing that the cellular structure remains relatively intact to contain bioactive components such as globular proteins or other water-soluble compounds within the cells. Preferred materials include embryos, larvae, neonates or adult cladocerans such as Daphnia, rotifers such as Brachionus, decapsulated Artemia cysts, nematodes, oligochaetes, polychaetes or insects.

In another aspect of the preferred embodiment, the size and shape of the bioactive food complex is made to complement the feeding mechanism and behavior of the aquatic animal target species. For example, in the case of larval suspension feeders, microcapsules or beads can be made by atomizing Emulsion-2 with nitrogen gas into a bath of about 5-20 weight percent calcium chloride and collecting gel microcapsules that range in size from about 20-200 mu. For larval and postlarval animals that feed on live prey or on detritus, microcapsules can be made that range in size from about 100-1000 mu. Wet sieving is used to collect microcapsules or beads of the desired size range.

For aquatic animals that can feed on worms, vermiform shapes or noodles can be made that resemble nematode, oligochaete or polychaete worms in a size of about 0.2 mm-20 mm (girth)×0.1 cm-25 cm (length). Noodle-like shapes are made by extruding Emulsion-2 into a bath of 5-20 weight percent calcium chloride. For animals such as sea urchins, snails, abalone or others that feed on aquatic vegetation, sheets can be made that range in size from about 0.1 cm-1.0 cm (thick)×1.0-100 cm (wide)×1.0 cm-100 cm (long). Sheets are made by using appropriately shaped molds (e.g., stainless steel or plastic) to form the gel before bathing in 5-20 weight percent calcium chloride.

The bioactive food complex can be preserved as a semi-solid paste without drying in about 10-25 weight percent solution of calcium chloride, sodium chloride, potassium chloride or other salts. A mold inhibitor such as glycerol, propylene glycol or propionic acid can be added to preserve the moist product. In another aspect of the invention, a cryopreservative such as dimethylsulfoxide (DMSO), glycerol, butylated hydroxytoluene (BHT), sucrose, raffinose, manitol, ethylene glycol, propylene glycol, methanol, 1,2-propanediol, 1,3-butanediol, 2,3-butendiol is used at about 1 to 50 weight percent of the final preparation before freezing the bioactive food complex at −5° C. or below.

The bioactive food complex can be stored as a moist material under normal room temperatures of about 25° C. without freezing or can be stored frozen with the aid of cryopreservatives when needed to preserve the structural integrity of soft-bodied invertebrate organisms, embryos or other bioactive cells that lack protective cell walls or rigid membranes.

In the control of disease the bioactive food complex delivers probiotic bacteria, enzymes for degradation or digestion of quorum sensing molecules and inhibitory furanone compounds as part of a feed product for aquatic animals. The bioactive food complex is added directly to the containment vessel of the aquatic species such as a larval rearing tank in a shrimp hatchery where it is eaten. Upon mastication and ingestion of the bioactive food complex the spores of the probiotic species will become hydrated and germinate to the active state. Probiotics delivered as dry vegetative cells will germinate more rapidly and start growing in the feed, gut and faeces and pseudo-faeces. Concurrently the inhibitory furanones will be released in the gut and subsequently into the containment system environment via faeces and pseudo-faeces. The probiotic Bacillus will grow and compete for space and food at the expense of pathogens and inhibit their growth by in situ production of antibiotics. Other strains of Bacillus included in the invention will produce enzymes that degrade or digest HSL and AHL signal molecules present in the microenvironment. The furanones will prevent the expression of virulence genes regulated by HSL and AHL in pathogens such as Vibrio.

The combined effects of probiotics and inhibitory furanones will provide the most effective control of disease in the hatchery environment and other aquatic environments. The bioactive food complex will provide essential micro and macronutrients required for normal growth and survival of larval shrimp and eliminate the need to use live and fresh foods.

In the case of application to conventional feeds for nursery and grow-out phases of aquaculture, Emulsion-1 of the present invention or the bioactive food complex can be included as top-dress coating or enrobing of the conventionally processed aquatic feeds such as pellets. In the case of using the bioactive food complex as the active ingredient of a top dress preparation, it is blended into the edible oil used as the top dress forming a suspension in the oil and then the suspension is sprayed or coated on to the pelleted feed before bagging. Alternatively, Emulsion-1 can be used directly as the top dress of the pelleted feed by spraying or coating the pellets directly with Emulsion-1.

U.S. Pat. No. 4,352,883 describes forming a suspension of living cells in sodium alginate and extruding the suspension dropwise into a bath of calcium alginate to form an immobilized cell complex in calcium alginate which is then further complexed with polylysine forming a polyelectrolyte complex that is more stable than the calcium alginate complex. Major differences between cell immobilization of the this patent and the present invention are:

(1) the material described in the patent is not a two-phase emulsion system but simply involves living cells suspended in hydrocolloid polymer before entrapment;

(2) the material described in the patent does not include nutrients of present invention formulated to provide nutrition as a food or feed for animals in addition to bioactive materials to be consumed directly by animals.

Sefton and Broughton Biochimica et Biophysica Acta, 7171,473 (1982) (Encyclopedia of Polymer Science and Engineering,/vol. 9, 2nd/ed. (1987)) formed a water-in-oil emulsion with cells dispersed in aqueous phase and with diethyl phthalte serving as the oil phase. This primary emulsion was emulsified in mineral oil whereby the mineral oil serves only to form a secondary emulsion. The major difference between this procedure and the present invention is that in the present invention the edible oil mixture serves (1) as a source of essential nutrients and bioactive materials to be consumed by an animal, (2) as a hydrophobic coating to prevent organic matter loss leaching into the aquatic environment, and (3) as the continuous phase of Emulsion-1.

U.S. Pat. No. 5,776,490 is a modification of the CXM concept with LWMs contained within complex, cross-linked protein microcapsules rather than within a hydrocolloid matrix of ionically crosslinked calcium alginate and gelatin. Emulsion-1 could be used to replace the LWMs of the invention.

### EXAMPLES

Having generally described this invention, a further understanding can be obtained by reference to certain specific examples which are provided herein for purposes of illustration only and are not intended to be limiting unless otherwise specified.

### Example 1

### Bioactive Food Complex Product and Control of Disease—Hatchery Feed

A bioactive food complex product is prepared according to the present invention by combining 2500 grams of a lipid mixture containing fish oil plus lipid-soluble ingredients including lipid-soluble bioactive compound such as inhibitory furanone as presented in Table 1. The lipid mixture is then mixed with 1000 grams of a feedstuffs mix containing probiotic bacteria, other bioactive compounds and dry powder nutrient compounds as presented in Table 1. The combination is vigorously mixed to form Emulsion-1, which in this example is a solids-in-oil emulsion with the lipid mixture forming the continuous phase and the feedstuff mix forming the dispersed phase. A second emulsion is prepared by combining Emulsion-1 with 5711 grams of a sodium alginate plus gelatin polymer prepared at pH 12 as presented in Table 1. The combination is vigorously mixed to form a second emulsion wherein Emulsion-1 is dispersed in the polymer serving as the continuous phase. The second emulsion is atomized into a bath of 20% w/w calcium chloride solution whereby alginate in the atomized droplets are ionically crosslinked with calcium to form a physically stable calcium-alginate matrix. The crosslinked gel droplets are collected by filtration forming a moist paste. The moist paste is preserved with 569 grams of a 25% w/w calcium chloride or sodium chloride solution plus 567 grams of propylene glycol plus 11 grams of an industrial gum to provide consistency to the semi-solid paste, as presented in Table-1. In the prevention of disease and to provide supplementary nutrition the resulting Bioactive Food Complex Product is fed to larval and post-larval shrimp. The probiotic bacteria of the invention present as part of the feedstuff mix germinate and grow in the water and the GI tract of shrimp where some of the selected strains produce antibiotic against pathogenic bacteria such as Vibrio and compete with the pathogens for space and nutrients while other selected strains of the invention produce enzymes that degrade or digest quorum sensing signal molecules of the pathogens. Concurrently, the inhibitory furanones, which are carried and delivered by the invention, inhibit quorum sensing processes regulated by signal molecules such as HSL and AHL of the pathogens. Disease is efficiently prevented by delivery of the bioactive compounds by the invention and by the application of these different mechanisms permitted by the invention.

TABLE 1
Example composition of Bioactive Food Complex Product.

| Bioactive Lipid Mixture | grams | Bioactive Feedstuff Mix | grams |
|---|---|---|---|
| Fish oil | 2316 | Liver powder | 510.0 |
| Inhibitory furanone | 20.0 | Squid extract | 49.0 |
| Antioxidant | 2.5 | Fish protein | 20.0 |
| Lecithin | 115.0 | Yeast extract | 160.0 |
| EmulsifIer | 25.0 | Probiotic bacteria | 150.0 |
| Cholesterol | 10.0 | Algae | 50.0 |
| Astaxanthin | 9.0 | Vitamin mix | 50.0 |
| Fat sol vit | 2.5 | Carrotenoid pigment | 10.0 |
| Total Lipid-phase | 2,500 | Trace minerals | 1.0 |
| | | Total Solids phase | 1,000 |
| Total Emulsion-1 | 3,500 | | |
| Polymer | grams | Paste Additives | grams |
| Sodium alginate | 143 | Salt solution | 569 |
| Gelatin | 29 | Preservative | 557 |
| Water | 5,539 | Gum | 11 |
| Total polymer | 5,711 | Total additives | 1,137 |
| Total Emulsion-2 | 9,211 | | |

### Example 2

Emulsion-1 comprises a bioactive food complex for application as a top-dress coating for conventional animal feeds such as those made by pelleting and extruding and is made by combining 2500 grams of a lipid mixture containing fish oil plus lipid-soluble ingredients including lipid-soluble bioactive compound such as inhibitory furanone as presented in Table 2. The lipid mixture is then mixed with 1000 grams of a feedstuffs mix containing probiotic bacteria and/or other bioactive compounds as presented in Table 2. The combination is vigorously mixed to form Emulsion-1, which in this example is a solids-in-oil emulsion with the lipid mixture forming the continuous phase and the feedstuff mix forming the dispersed phase. The entire emulsion is then applied by spraying or coating at a concentration of about 1 to 300 kg of oil emulsion per metric ton of animal feed with a preferred concentration of about 10 to 80 kg per metric ton of feed. In this example a second emulsion is not needed to apply bioactive compounds of the invention to conventional pelleted or extruded or other feeds, which are already formed and can be coated with Emulsion-1. In the prevention of disease, the conventional feeds, which have been coated with the bioactive emulsion of the invention, are fed to aquatic livestock such as shrimp or fish. The probiotic bacteria of the invention that are applied to the feed as part of the feedstuff mix germinate and grow in the water and the GI tract of shrimp where some of the selected strains produce antibiotics against pathogenic bacteria such as Vibrio and compete with the pathogens for space and nutrients while other selected strains of the invention produce enzymes that degrade or digest quorum sensing signal molecules of the pathogens such as HSL and AHL. Concurrently, the inhibitory furanones, which are carried and delivered by Emulsion-1 of the invention, inhibit quorum sensing processes regulated by signal molecules such as HSL and AHL of the pathogens. Disease is efficiently prevented by delivery of the bioactive compounds by the invention and by the application of these different mechanisms permitted by the invention.

TABLE 2
Example composition of Emulsion-1 for use as
bioactive top dress coating of pelleted feeds.

| Bioactive Lipid | grams |
|---|---|
| Fish oil | 2107 |
| Inhibitory furanone | 25 |
| Antioxidant | 3 |
| Lecithin | 115 |
| Emulsifier | 25 |
| Total Lipid-phase | 2,500 |
| Bioactive Feedstuff | grams |
| Yeast extract | 250.0 |
| Probiotic bacteria | 750.0 |
| Total Solids phase | 1,000 |
| Total Emulsion-1 | 3,500 |

Obviously, numerous modifications and variations of the present invention are possible in light of the above teachings. It is therefore to be understood that within the scope of the appended claims, the invention may be practiced otherwise than as specifically described herein.

1. A method of controlling and/or preventing diseases in aquatic animals, comprising feeding the aquatic animals a composition comprising at least one probiotic bacteria and at least one inhibitory or regulatory compound.

2. The method of claim 1 wherein the probiotic bacteria and the inhibitory or regulatory compound are incorporated into an animal feed, which is added to the water containing the aquatic animal.

3. The method of claim 1 wherein the probiotic bacteria and inhibitory or regulatory compound are incorporated into an animal feed by incorporating them as Emulsion-1 of the bioactive food complex, which is used as a top dress coating for the animal feed.

4. The method of claim 1 wherein the probiotic bacteria and inhibitory or regulatory compounds is incorporated into a feed for aquatic animal via incorporation into the bioactive food complex that is used as a complete feed and added directly to the containment systems of the aquatic animals such an aquaria, tanks, cages, raceways, ponds or other enclosures.

5. The method of claim 1 wherein the probiotic bacteria and inhibitory or regulatory compounds is applied to aquatic animal feeds by incorporating them as Emulsion-1 of the bioactive food complex used as a top dress coating for the aquatic animal feed.

6. The method of claim 1 wherein the probiotic bacteria are selected strains of bacteria including but not limited to strains of Bacillus subtilis, Bacillus laterosporus, B. licheniformis, Bacillus azotoformans, Bacillus circulans, Bacillus pumilus, Bacillus firmus, Paenibacillus polymyxa, Paenibacillus macerans, Alteromonas sp.

7. The method of claim 1 wherein the inhibitory compounds are furanones or other lipid soluble bioactive compounds.

8. The method of claim 1 wherein the pathogens which are controlled are Gram negative bacteria including but not limited to Vibrio harveyi, V. parahaemolyticus, V. splendidus, V. mimicus, V cholerae, V alginolyticus, V. anguillarum, other Vibrio sp., Aeromonas sp.

9. The method of claim 1 wherein the pathogens which are controlled are Gram positive pathogens, including but not limited to Streptococcus, Camobacterium, Micrococcus and others.

10. The method of claim 1 wherein control of pathogens takes place in the digestive tracts of the animals.

11. The method of claim 1 wherein control of pathogens takes place in the environment of the animals including the feed bins, feed trays, pens, stalls, aquaria, tanks, cages, raceways, ponds, and in the water, surfaces and sediments of these and other enclosures.

12. A method for forming a bioactive food complex comprising:
forming a first emulsion (Emulsion-1) comprising a solids-in-oil or an oil-in-solids emulsion of bioactive materials and powder nutrients forming the solid phase and lipid soluble bioactive compounds dissolved in edible oil forming the oil phase and of a second emulsion comprising an oil-in-polymer or solids-in-polymer emulsion with the dispersed phase comprising Emulsion-1 and a hydrocolloid polymer serving as the continuous phase, and
exposing said hydrocolloid polymer to ions, thereby ionically crosslinking the polymer forming a physically stable gel matrix, entraping Emulsion-1 in the second emulsion, thereby forming the bioactive food complex.

13. The method of claim 12 wherein the oil phase of the first emulsion is a member selected from the group consisting of fish oil, a refined fish oil and a vegetable oil and contains lipid soluble bioactive compounds.

14. The method of claim 13 wherein the lipid soluble bioactive compound is a inhibitory anfuranones and the oil is a member of the group consisting of menhaden fish oil, salmon oil, anchovy oil, sardine oil, tuna oil, mackerel oil, capeline oil, squid oil, pollack oil, cod oil, dietary fish oil supplement, soybean oil, safflower, corn oil, and palm-kernel oil.

15. The method of claim 12 wherein the continuous phase polymer of the second emulsion is composed of about 0.5-4.0 weight percent polymer, said polymer being a member selected from the group consisting of alginate and carrageenan polymers.

16. The method of claim 12 wherein the continuous phase polymer of the second emulsion is formed in deionized water at about 45° C.-85° C. by the addition of at least one ion from the group consisting of sodium, potassium and calcium ions.

17. The method of claim 12 wherein the polymer is blended with a member selected from the group consisting of gelatin, zein, polylysine, polyarginine, chitosan, gum accacia, and locust bean gum preparations at about 0.1-3.5 weight percent in water.

18. The method of claim 12 wherein the solids to oil ratio is between about 0.01:1 to 100:1 preferably 0.1:1 to 3:1 by weight.

19. The method of claim 13 wherein the oil has added thereto at least one member of the group consisting of lecithin, cholesterol, and emulsifying agent, and antioxidant, a fat soluble vitamin, and astaxanthin.

20. The method of claim 13 wherein the oil comprises a mixture including edible oil, about 0.0001 to 50% lipid soluble bioactive compounds such as inhibitory furanones, 0.5 to 5% emulsifying agent, about 1 to 10% lecithin, about 1 to 10% cholesterol, and about 0.01 to 0.5% antioxidant, by weight.

21. The method of claim 20 wherein the oil includes per KG. about 2,000 to 8,000 IU vitamin A, about 1,000 to 4,000 IU vitamin D₃, about 2,000 to 8,000 IU vitamin E, about 20 to 80 mg vitamin K, and about 2,000 mg astaxanthin.

22. The method of claim 13 wherein the oil includes in excess of 1 weight percent omega-3 fatty acid, in excess of 0.1 weight percent eicosapentaenoic acid and in excess of 0.1 weight percent docosahexaenoic acid.

23. The method of claim 13 wherein the oil is warmed to about 25 to 40° C. prior to the addition of said bioactive materials and powdered nutrient.

24. The method of claim 12 wherein said bioactive materials and powdered nutrient is at least one member selected from the group consisting of bacterial spores, bacterial cell walls, bacterial cells, yeast cell walls, yeast extract, dietary yeast, brewers yeast, yeast cells, and algal cells.

25. The method of claim 24 wherein said bacterial cells is at least one member selected from the group consisting of Bacillus, Lactobacillus, Streptococcus, Bifidobacterium, Leuconostor, and Alteromonad cells.

26. The method of claim 12 wherein oil phase prior to adding the powder nutrient feedstuffs and the bioactive materials has a concentration of total omega-3 fatty acids greater than about 1 weight percent, eicosapentaenoic acid (EPA) greater than about 0.1 weight percent, and docosahexaenoic acid (DHA) greater than about 0.1 weight percent.

27. The method of claim 24 wherein said algal cells is at least one member selected from the group consisting of algal cells including but not limited to Haematococcus, Schizochytrium, Dunelliela, Chaetoceros, Tetraselmis, Skeletonema, Nannochloropsis, Thalassiosira, Phaeodactylum, Isochrysis, Pavlova.

28. The method of claim 12 wherein the solid phase of the first emulsion comprises a solid material selected from the group consisting of bacterial material, yeast material, algal material, medicament material, animal protein products, plant protein products, and poultry egg products.

29. The method of claim 12 wherein the solid phase of the first emulsion comprises a solid material selected from the group consisting of bacterial material selected from the group consisting of bacterial spores, bacterial cell walls, and bacterial cells; yeast material selected from the group consisting of yeast cell walls, yeast extract, dietary yeast, brewer's yeast, yeasts cells, algal material, medicaments, powder feedstuffs, animal protein products, plant protein products, poultry egg products, cyanocobalamin, D-biotin, D-pantothenic acid, folic acid, L-ascorbyl-2polyphosphate, myoinositol, niacin, p-amino-benzoic acid, pyridoxine hydrochloride, riboflavin, thiamine hydrochloride, and choline chloride.

30. The method of claim 29 wherein the solid phase of the first emulsion comprises a bacterial material selected from the group consisting of Lactobacillus, Bacillus, Streptococcus, Bifidobacterium, Leuconostor, and Alteromonad,

31. The method of claim 30 wherein the bacterial material is present at about 0.1-95 weight percent of the solid phase.

32. The method of claim 28 wherein the solid phase of the first emulsion comprises a yeast material selected from the group consisting of torrula yeast and Phaffia yeast at about 0.1-50 weight percent.

33. The method of claim 28 wherein the yeast material is present at about 0.1-50 weight percent of the solid phase.

34. The method of claim 27 wherein the solid phase of the first emulsion comprises a algal material selected from the group consisting of Haematococcus, Schizochytrium, Dunelliela, Chaetoceros, Tetraselmis, Skeletonema, Nannochloropsis, Thalassiosira, Phaeodactylum, Isochrysis, and Pavlova.

35. The method of claim 34 wherein the yeast material is present at about 1-80 weight percent of the solid phase.

36. The method of claim 31 wherein the solid phase of the first emulsion comprises antibiotics selected from the group consisting of Sarafin, Romet and Terramycin,

37. The method of claim 36 wherein the antibiotics is present at about 0.01-50 weight percent of the solid phase.

38. The method of claim 31 wherein the solid phase of the first emulsion comprises animal protein products at about 0-95 weight percent of the solid phase.

39. The method of claim 31 wherein the solid phase of the first emulsion comprises plant protein products at about 0-95 weight percent of the solid phase.

40. The method of claim 31 wherein the solid phase of the first emulsion comprises poultry egg products at about 0-25 weight percent of the solid phase.

41. The method of claim 29 wherein the solid phase of the first emulsion comprises cyanocobalamin at about 40-60 mg/kg; D-biotin at about 5-20 mg/kg; D-pantothenic acid at about 250-350 mg/kg; folic acid at about 10-30 mg/kg; L-ascorbyl-2polyphosphate (STAY-C, stable form of vitamin C) at about 1,000-4 000 mg/kg; myo-inositol at about 3,000-4,000 mg/kg; niacin at about 600-800 mg/kg; p-amino-benzoic acid at about 350-450 mg/kg; pyridoxine hydrochloride at about 40-60 mg/kg; riboflavin at about 125-175 mg/kg; thiamine hydrochloride at about 50-80 mg/kg; and choline chloride at about 6,500-7,500 mg/kg.

42. The method of claim 12 wherein relatively fragile bioactive components are embedded in the second emulsion.

43. The method of claim 42 wherein said relatively fragile bioactive components are a member selected from the group consisting of invertebrate embryos and invertebrate organisms.

44. The method of claim 42 wherein said relatively fragile bioactive components are a member selected from the group consisting of live metazoan, frozen metazoan, lyophilized metazoan, live protozoan, frozen protozoan, lyophilized protozoan, live animal or plant tissue, frozen animal or plant tissue and lyophilized plant or animal tissue.

45. The method of claim 42 wherein said relatively fragile bioactive components are a member selected from the group consisting of embryos, larvae, neonates and adult cladocerans.

46. The method of claim 41 wherein said relatively fragile bioactive components are a member selected from the group consisting of Daphnia, rotifers, decapsulated Artemia cysts, nematodes, oligochaetes, polychaetes and insects.

47. The method of claim 46 wherein said rotifiers are Brachionus.

48. A bioactive food complex for feeding aquatic animals, comprising of a first emulsion (Emulsion-1) that is a solids-in-oil or an oil-in-solids emulsion of bioactive materials and powder nutrients that form the solid phase and lipid soluble bioactive compounds dissolved in edible oil that form the oil phase and of a second emulsion comprised of an oil-in-polymer or solids-in-polymer emulsion with the dispersed phase comprised of Emulsion-1 and a hydrocolloid polymer serves as the continuous phase; the complex is exposed to ions whereby the hydrocolloid polymer is ionically crosslinked and forms a physically stable gel matrix, with Emulsion-1 entrapped in the second emulsion, thereby comprising the bioactive food complex.

49. The bioactive food complex of claim 48 wherein the continuous phase polymer of the second emulsion is composed of about 0.5-4.0 weight percent polymer, said polymer being a member selected from the group consisting of alginate and carrageenan polymers and wherein the polymer is crosslinked by the addition of a member selected from the group consisting of sodium, potassium and calcium ions.

50. The bioactive food complex of claim 48 wherein the oil phase of the first emulsion is a member selected from the group consisting of fish oil, refined fish oil and vegetable oil.

51. The method of claim 50 wherein the fish oil is a member selected from the group consisting of menhaden fish oil, salmon oil, anchovy oil, sardine oil, tuna oil, mackerel oil, capeline oil, squid oil, pollack oil, cod oil, dietary fish oil supplement, soybean oil, safflower, corn oil, and palm-kernel oil.

52. The bioactive food complex of claim 48 wherein the solids to oil ratio is between about 0.01:1 to 100:1, preferably 0.1:1 to 3:1 by weight.

53. The bioactive food complex of claim 52 wherein the oil has added thereto at least one member selected from the group consisting of lecithin, cholesterol, and emulsifying agent, and antioxidant, a fat soluble vitamin, and astaxanthin.

54. The bioactive food complex of claim 48 wherein the oil comprises a mixture including edible oil, about 0.5 to 5% emulsifying agent, about 1 to 10% lecithin, about 1 to 10% cholesterol, and about 0.01 to 0.5% antioxidant, by weight.

55. The bioactive food complex of claim 48 wherein the oil includes per KG about 2,000 to 8,000 IU vitamin A, about 1,000 to 4,000 IU vitamin D₃, about 2,000 to 8,000 IU vitamin E, about 20 to 80 mg vitamin K, and about 2,000 mg astaxanthin.

56. The bioactive food complex of claim 48 wherein the oil includes in excess of 1 weight percent omega-3 fatty acid, in excess of 0.1 weight percent eicosapentaenoic acid and in excess of 0.1 weight percent docosahexaenoic acid.

57. The bioactive food complex of claim 48 wherein the oil is warmed to about 25 to 40° C. prior to the addition of said bioactive materials and powdered nutrient.

58. The bioactive food complex of claim 48 wherein said bioactive material is at least one member selected from the group consisting of bacterial spores, bacterial cell walls, bacterial cells, yeast cell walls, yeast extract, dietary yeast, brewers yeast, yeast cells, and algal cells.

59. The bioactive food complex of claim 58 wherein said bacterial cells comprise at least one member selected from the group consisting of Lactobacillus, Bacillus, Streptococcus, Bifidobacterium, Leuconostor, and Alteromonad cells.

60. The bioactive food complex of claim 58 wherein the bioactive materials has a concentration of total omega-3 fatty acids greater than about 1 weight percent, eicosapentaenoic acid (EPA) greater than about 0.1 weight percent, and docosahexaenoic acid (DHA) greater than about 0.1 weight percent.

61. The bioactive food complex of claim 58 wherein said algal cells comprise at least one member selected from the group consisting of algal cells including but not limited to Haematococcus, Schizochytrium, Dunelliela, Chaetoceros, Tetraselmis, Skeletonema, Nannochloropsis, Thalassiosira, Phaeodactylum, Isochrysis, Pavlova.

62. The bioactive food complex of claim 48 wherein the solid phase of the first emulsion comprises a solid material selected from the group consisting of bacterial material, yeast material, algal material, medicament material, animal protein products, plant protein products, and poultry egg products.

63. The bioactive food complex of claim 48 wherein the solid phase of the first emulsion comprises a solid material selected from the group consisting of bacterial material selected from the group consisting of bacterial spores, bacterial cell walls, and bacterial cells; yeast material selected from the group consisting of yeast cell walls, yeast extract, dietary yeast, brewer's yeast, yeasts cells; algal material selected from the group consisting of algal cell preparations; medicaments; powder feedstuffs selected from the group consisting of animal protein products; plant protein products; poultry egg products; cyanocobalamin; D-biotin; D-pantothenic acid; folic acid; L-ascorbyl-2polyphosphate; myo-inositol; niacin; p-amino-benzoic acid; pyridoxine hydrochloride; riboflavin; thiamine hydrochloride; and choline chloride.

64. The bioactive food complex of claim 48 wherein the solid phase of the first emulsion comprises a bacterial material selected from the group consisting of Lactobacillus, Bacillus, Streptococcus, Bifidobacterium, Leuconostor, and Alteromonad,

65. The bioactive food complex of claim 64 wherein the bacterial material is present at about 0.1-95 weight percent of the solid phase.

66. The bioactive food complex of claim 48 wherein the solid phase of the first emulsion comprises a yeast material selected from the group consisting of torrula yeast and Phaffia yeast at about 0.1-50 weight percent.

67. The bioactive food complex of claim 66 wherein the yeast material is present at about 0.1-50 weight percent of the solid phase.

68. The method of claim 48 wherein the solid phase of the first emulsion comprises a algal material selected from the group consisting of Haematococcus, Schizochytrium, Dunelliela, Chaetoceros, Tetraselmis, Skeletonema, Nannochloropsis, Thalassiosira, Phaeodactylum, Isochrysis, Pavlova.

69. The bioactive food complex of claim 67 wherein the yeast material is present at about 1-80 weight percent of the solid phase.

70. The bioactive food complex of claim 68 wherein the solid phase of the first emulsion comprises antibiotics selected from the group consisting of Sarafin, Romet, and Terramycin.

71. The bioactive food complex of claim 69 wherein the antibiotics is present at about 0.01-50 weight percent of the solid phase.

72. The bioactive food complex of claim 62 wherein the solid phase of the first emulsion comprises animal protein products at about 0-95 weight percent of the solid phase.

73. The bioactive food complex of claim 62 wherein the solid phase of the first emulsion comprises plant protein products at about 0-95 weight percent of the solid phase.

74. The bioactive food complex of claim 62 wherein the solid phase of the first emulsion comprises poultry egg products at about 0-25 weight percent of the solid phase.

75. The bioactive food complex of claim 62 wherein the solid phase of the first emulsion comprises cyanocobalamin at about 40-60 mg/kg; D-biotin at about 5-20 mg/kg; D-pantothenic acid at about 250-350 mg/kg; folic acid at about 10-30 mg/kg; L-ascorbyl-2polyphosphate (STAY-C, stable form of vitamin C) at about 1,000-4,000 mg/kg; myo-inositol at about 3,000-4,000 mg/kg; niacin at about 600-800 mg/kg; p-amino-benzoic acid at about 350-450 mg/kg; pyridoxine hydrochloride at about 40-60 mg/kg; riboflavin at about 125-175 mg/kg; thiamine hydrochloride at about 50-80 mg/kg; and choline chloride at about 6 500-7,500 mg/kg.

76. The bioactive food complex of claim 48 wherein relatively fragile bioactive components are embedded in the second emulsion.

77. The bioactive food complex of claim 76 wherein said relatively fragile bioactive components are a member of the group consisting of invertebrate embryos and invertebrate organisms.

78. The bioactive food complex of claim 76 wherein said relatively fragile bioactive components are a member of the group consisting of live metazoan, frozen metazoan, lyophilized metazoan, live protozoan, frozen protozoan, lyophilized protozoan, live animal or plant tissue, frozen animal or plant tissue and lyophilized plant or animal tissue.

79. The bioactive food complex of claim 76 wherein said relatively fragile bioactive components are a member of the group consisting of embryos, larvae, neonates and adult cladocerans.

80. The bioactive food complex of claim 76 wherein said relatively fragile bioactive components are a member of the group consisting of Daphnia, rotifers, decapsulated Artemia cysts, nematodes, oligochaetes, polychaetes and insects.

81. The bioactive food complex of claim 80 wherein said rotifiers are Brachionus.

82. A bioactive food complex for feeding aquatic animals, said complex comprising: a first emulsion comprising a solids-in-oil or oil-in-solids emulsion (Emulsion-1) of bioactive materials and powder nutrient forming the solid phase and edible oil forming the oil phase, said oil being a member selected from the group consisting of fish oil, a refined fish oil and a vegetable oil, said the solids to oil ratio is between about 0.01:1 to 100:1 preferably 0.1:1 to 3:1 by weight; and a second emulsion comprising Emulsion-1 dispersed in a hydrocolloid polymer, said Emulsion-1 dispersed in said second emulsion, said a hydrocolloid polymer serving as the continuous phase, said hydrocolloid polymer being ionically crosslinked to form a physically stable matrix, said hydrocolloid continuous phase including dispersed invertebrate organisms.

83. The bioactive food complex of claim 82 wherein said fish oil is a member of the group consisting of menhaden fish oil, salmon oil, anchovy oil, sardine oil, tuna oil, mackerel oil, capeline oil, squid oil, pollack oil, cod oil, dietary fish oil supplement, soybean oil, safflower, corn oil, and palm-kernel oil.

84. The bioactive food complex of claim 83 wherein the fish oil has added thereto at least one member of the group consisting of lecithin, cholesterol, emulsifying agent, antioxidant, fat soluble vitamin, and astaxanthin.

85. The bioactive food complex of claim 82 wherein the oil comprises a mixture including edible oil, about 0.0001 to 50% lipid soluble bioactive compounds such as inhibitory furanones, about 0.5 to 5% emulsifying agent, about 1 to 10% lecithin, about 1 to 10% cholesterol, and about 0.01 to 0.5% antioxidant, by weight.

86. The bioactive food complex of claim 82 wherein said bioactive material comprises at least one member selected from the group consisting of bacterial spores, bacterial cell walls, bacterial cells, yeast cell walls, yeast extract, dietary yeast, brewers yeast, yeast cells, and algal cells.

87. The bioactive food complex of claim 86 wherein said bacterial cells comprise at least one member selected from the group consisting of Lactobacillus, Bacillus, Streptococcus, Bifidobacterium, Leuconostor, and Alteromonad cells.

88. The bioactive food complex of claim 86 wherein the bioactive materials has a concentration of total omega-3 fatty acids greater than about 1 weight percent, eicosapentaenoic acid (EPA) greater than about 0.1 weight percent, and docosahexaenoic acid (DHA) greater than about 0.1 weight percent.

89. The bioactive food complex of claim 86 wherein said algal cells comprise at least one member selected from the group consisting of algal cells including but not limited to Haematococcus, Schizochytrium, Dunelliela, Chaetoceros, Tetraselmis, Skeletonema, Nannochloropsis, Thalassiosira, Phaeodactylum, Isochrysis, and Pavlova.

90. The bioactive food complex of claim 82 wherein the solid phase of the first emulsion comprises a solid material selected from the group consisting of bacterial material, yeast material, algal material, medicament material, animal protein products, plant protein products, and poultry egg products.

91. The bioactive food complex of claim 82 wherein the solid phase of the first emulsion comprises a solid material selected from the group consisting of bacterial material selected from the group consisting of bacterial spores, bacterial cell walls, and bacterial cells; yeast material selected from the group consisting of yeast cell walls, yeast extract, dietary yeast, brewer's yeast, yeasts cells; algal material selected from the group consisting of algal cell preparations; medicaments; powder feedstuffs selected from the group consisting of animal protein products; plant protein products; poultry egg products; cyanocobalamin; D-biotim; D-pantothenic acid; folic acid; L-ascorbyl-2polyphosphate; myo-inositol; niacin; p-amino-benzoic acid; pyridoxine hydrochloride; riboflavin; thiamine hydrochloride; and choline chloride.

92. The bioactive food complex of claim 91 wherein hydrocolloid continuous phase further includes dispersed invertebrate organisms.
